SEQUENCE LISTING (14 sequences)

SEQ ID NO: 1 - DNA, 9 bp, Homo sapiens
tctgagtca

SEQ ID NO: 2 - DNA, 147 bp, Homo sapiens
agataactgggccaaccatgactcagtgcttctggaggccaacaggacttctgagtcatcctgtgggggtggaggtgggacaagggaaaggggtgaatggtactgctgattacaacctctggtgctgcctccccctcctgtttatct

SEQ ID NO: 3 - DNA, 356 bp, Homo sapiens
tcgaccctctggaacctatcagggaccacagtcagccaggcaagcacatctgcccaagccaagggtggaggcatgcagctgtgggggtctgtgaaaacacttgagggagcagataactgggccaaccatgactcagtgcttctggaggccaacaggacttctgagtcatcctgtgggggtggaggtgggacaagggaaaggggtgaatggtactgctgattacaacctctggtgctgcctccccctcctgtttatctgagagggaaggccatgcccaaagtgttcacagccaggcttcaggggcaaagcctgacccagacagtaaatacgttcttcatctggagctgaagaaattc

SEQ ID NO: 4 - DNA, 26 bp, Artificial Sequence
  Synthetic PCR primer
tgaaggtcggtgtgaacggatttggc

SEQ ID NO: 5 - DNA, 24 bp, Artificial Sequence
  Synthetic PCR primer
catgtaggccatgaggtccaccac

SEQ ID NO: 6 - DNA, 18 bp, Artificial Sequence
  Synthetic PCR primer
gtccctgctcctggcttt

SEQ ID NO: 7 - DNA, 20 bp, Artificial Sequence
  Synthetic PCR primer
atgcggagcagctccaggtt

SEQ ID NO: 8 - DNA, 20 bp, Artificial Sequence
  Synthetic PCR primer
catcagcgtttggatgcctt

SEQ ID NO: 9 - DNA, 18 bp, Artificial Sequence
  Synthetic PCR primer
tgggcaggctgctggtta

SEQ ID NO: 10 - DNA, 21 bp, Artificial Sequence
  Synthetic PCR primer
ttagtggtacttgtgagccaa

SEQ ID NO: 11 - DNA, 20 bp, Artificial Sequence
  Synthetic PCR primer
ctgatgaagaatgagagagc

SEQ ID NO: 12 - DNA, 23 bp, Artificial Sequence
  Synthetic PCR primer
tagaggtacttctcatcagtcag

SEQ ID NO: 13 - DNA, 20 bp, Artificial Sequence
  Synthetic PCR primer
ctgataagaaacaccacccc

SEQ ID NO: 14 - DNA, 20 bp, Artificial Sequence
  Synthetic PCR primer
atgcggagcagctcctggtt

(19) United States
(12) Patent Application Publication
(10) Pub. No.: US 2002/0148000 A1
(43) Pub. Date: Oct. 10, 2002

(54) HS-40 enhancer-containing vector in transgenic animals

(76) Inventor: Che-Kun James Shen, Taipei (TW)

Correspondence Address: Michael R. Ward, Morrison & Foerster LLP, 425 Market Street, San Francisco, CA, 94105-2482, US

(21) Appl. No.: 10/014,220

(22) Filed: Nov. 9, 2001

Related U.S. Application Data
(63) Continuation-in-part of application No. 09/961,563, filed on Sep. 20, 2001.
Application No. 09/961,563 is a continuation of application No. 09/536,094, filed on Mar. 24, 2000.
Application No. 09/536,094 is a division of application No. 09/205,015, filed on Dec. 4, 1998, now abandoned.

(51) Int. Cl. A01K 67/027

(52) U.S. Cl. 800/14; 800/15; 800/16; 800/17; 800/18; 800/19

(57) ABSTRACT

A transgenic animal having a viral expression vector having a nucleic acid which includes (1) a transcriptional start site; (2) a promoter operably linked to the transcriptional start site; and (3) an enhancer operably linked to the promoter, the enhancer comprising the DNA sequence of SEQ ID NO:1 or the RNA equivalent thereof. The vector us useful for making transgenic animals. The transgenic animal may include pig, rat, cow, rabbit, goat, guinea pig, prairie baboon, squirrel, monkey, chimpanzee, bird, frog, toad, chicken, turkey and sheep.

### RELATED APPLICATIONS

This application is a continuation-in-part application of Ser. No. 09/961,563 filed Sep. 20, 2001, which is a continuation application of Ser. No. 09/536,094 filed Mar. 24, 2000, which is a divisional application of Ser. No. 09/205,015 filed Dec. 4, 1998 (abandoned). The disclosures of each of these prior applications are hereby incorporated by reference.

### FIELD OF THE INVENTION

This invention is in the field of transgenic animals.

### BACKGROUND OF THE INVENTION

Transgenic animals are of tremendous value to the scientific research community. In addition to their use in research, transgenic animals are useful for improving the efficiency of producing livestock products, producing drugs including antibodies and in the study of genetic diseases.

Gene expression in transgenic animals is often limited by the position in the genome where the transgene is integrated and by the number of copies of the transgene which have been integrated.

While transgenic animal technology has improved in recent years, there is still a tremendous need for position-independent and copy number dependent expression of transgenics in transgenic animals.

### SUMMARY OF THE INVENTION

The invention is based on the discovery that a single nucleotide change in the 3′NF-E2/AP1 element of the human HS-40 enhancer HS-40(mt), unlike the wild type HS-40 enhancer, confers position-independent and copy number-dependent expression on a transgene in transgenic animals. In addition, the single nucleotide change allows expression of the gene in the cells of transgenic animals, an effect not seen for the wild type HS-40 enhancer.

Accordingly, the invention features a viral expression vector (e.g., a retrovirus) having a nucleic acid including (1) a transcriptional start site; (2) a promoter (e.g., a tissue-specific promoter such as a ζ-globin promoter) operably linked to the transcriptional start site; and (3) an enhancer operably linked to the promoter, the enhancer including the mutated NF-E2/AP1 (mtNF-E2/AP1) DNA sequence TCTGAGTCA (SEQ ID NO:1) or the RNA equivalent thereof. The underlined “T” represents a mutation of the wild type “G” in the wild type NF-E2/AP1 (wtNF-E2/AP1) sequence. In a specific embodiment, the enhancer includes the minimal mutated HS-40 DNA sequence

| | |
|---|---|
| AGATAACTGGGCCAACCATGACTCAGTGCTTCTGGAGGCCAACA | (SEQ ID NO: 2) |
| GGACTTCTGAGTCATCCTGTGGGGGTGGAGGTGGGACAAGGGAAAGGGGTGA | |
| ATGGTACTGCTGATTACAACCTCTGGTGCTGCCTCCCCCTCCTGTTTATCT | |

or an RNA equivalent thereof. The bold sequence represents the mtNF-E2/AP1 site with the G to T mutation underlined. The minimal HS-40 enhancer sequence excludes a 5′GATA-1(b) site because it has been shown that this site is not necessary for HS-40 enhancer activity (Zhang et al., J Biol Chem 270:8501-8505, 1995).

The enhancer can also include the full mutated HS-40 enhancer sequence:

| | |
|---|---|
| TCGACCCTCTGGAACCTATCAGGGACCACAGTCAGCCAAGGCAAG | (SEQ ID NO: 3) |
| CACATCTGCCCAAGCCAAGGGTGGAGGCATGCAGCTGTGGGGGTCTGTGAAA | |
| ACACTTGAGGGAGCAGATAACTGGGCCAACCATGACTCAGTGCTTCTGGAGGC | |
| CAACAGGACTTCTGAGTCATCCTGTGGGGGTGGAGGTGGGACAAGGGAAAGG | |
| GGTGAATGGTACTGCTGATTACAACCTCTGGTGCTGCCTCCCCCTCCTGTTTAT | |
| CTGAGAGGGAAGGCCATGCCCAAAGTGTTCACAGCCAGGCTTCAGGGGCAAA | |
| GCCTGACCCAGACAGTAAATACGTTCTTCATCTGGAGCTGAAGAAATTC | |

or an RNA equivalent thereof. The bold sequence represents the mtnf-E2/AP1 site with the G to T mutation underlined. This sequence is referred to herein as the HS-40(mt) sequence, which differs from the wild type HS-40 [HS-40(wt)] sequence by the G/T mutation indicated above. Again, the single mutation is underlined. The vector can also contain a transcriptional termination signal (e.g., a polyadenylation signal). In other embodiments, the promoter drives transcription of a mRNA encoding a polypeptide (e.g., a growth hormone), the transcription beginning from the transcriptional start site.

A promoter is a nucleotide sequence required to facilitate transcription from a transcriptional start site, which is the site at which the first nucleotide of the transcript is transcribed, the nucleotide being complementary to the corresponding nucleotide in the nucleic acid. A promoter operably linked to a transcriptional start site means that the promoter is capable of driving transcription from the transcriptional start site in the absence of further nucleotide sequences.

An enhancer is a nucleic acid sequence which increases the level of transcription from a promoter. Enhancers need not be in any specified position in the nucleic acid in relation to the promoter, transcriptional start site, or transcriptional termination site. All that is required for a specific enhancer to be operably linked to a specific promoter is that the presence of the enhancer increases transcription driven by that promoter.

A transcriptional termination signal is a nucleic acid sequence which terminates transcription of a transcript. A variety of promoters, enhancers, and transcriptional termination signals are known in the art.

A viral expression vector is any combination of a nucleic acid and at least one protein which is useful for delivering a nucleic acid into a cell so as to express a transcript encoded by the nucleic acid in the cell. Other components, such as a lipid bilayer can also be present in the vector. An example of a viral expression vector is a retrovirus.

The invention also includes a transgenic non-human animal (e.g., a mouse or other rodent, pig, rat, cow, rabbit, goat, guinea pig, prairie, baboon, squirrel, monkey, chimpanzee, bird, amphibian, (frog, toad) chicken, turkey, or sheep) whose somatic and germ line cells contain at least one copy of a transgene comprising (1) a transcriptional start site; (2) a promoter (e.g., a tissue-specific promoter such as a ζ-globin promoter) operably linked to the open reading frame; and (3) an enhancer operably linked to the promoter. The enhancer includes the nucleotide sequence of SEQ ID NO:1 (e.g., SEQ ID NO:2). The transgenic animal expresses a transcript driven by the promoter, where the level of expression in at least one cell type (e.g., a erythroblast) of the animal is proportionally dependent on the copy number of the transgene, i.e., the greater the copy number, the greater the expression. Such a transcript can be an mRNA encoding anagriculturally or biomedically imported protein polypeptide (e.g., a growth hormone, or other proteins such as those described in PCT Publication WO 00/69900, which is hereby incorporated by reference in its entirety). In other embodiments, the somatic and germ line cells contain more than 5 copies (e.g., more than 15 copies) of the transgene.

The invention also features a method of expressing a transcript in an animal (e.g., a mouse or other rodent, pig, rat, rabbit, goat, guinea pig, prairie, baboon, squirrel, monkey, chimpanzee, bird, amphibian, cow, chicken, turkey, or sheep) by administering to the animal a nucleic acid comprising (1) an transcriptional start site for the transcript; (2) a promoter (e.g., a tissue-specific promoter such as a ζ-globin promoter) operably linked to the transcriptional start site; and (3) an enhancer operably linked to the promoter, the enhancer comprising the DNA sequence of SEQ ID NO:1 or 2 or the RNA equivalent thereof. The transcript can be a mRNA encoding a polypeptide. The polypeptide can be an agriculturally or biomedically important protein as described above. The nucleic acid can be administered by parental injection (e.g., intramuscular injection) or via a viral expression vector. The nucleic acid can further include a transcriptional termination signal (e.g., a polyadenylation signal).

Nucleic acids and viral vectors containing an enhancer having the mtNF-E2/AP1 sequence described above can be used to express a therapeutic antisense RNA or mRNA encoding a therapeutic polypeptide in an animal in a position-independent and transgene copy number dependent manner. This was an unexpected result because, previously, transgene expression was limited by position-effect variegation, silencing of transgenes, and the inability to increase expression by increasing the copy number of the transgene. See, e.g., Sabl et al., Genetics 142:447-458, 1996; Palmer et al., Sharpe et al., EMBO J 11:4565-4572, 1992; and Chen et al., Proc. Nati. Acad. Sci. USA 94:5798-5803,1997. By inclusion of an enhancer containing the mtNF-E2/AP1 sequence in the transgene sequence, these deficiencies in transgene expression are removed. Enhancement of transgene expression can result in transgenic animal models exhibiting more severe symptoms so that therapeutic efficacy in those models can be measured in a wider range of symptom severity. Examples of such models, which can be improved by the present invention, are described in U.S. Pat. Nos. 5,811,634 and 5,675,060

### BRIEF DESCRIPTION OF THE DRAWINGS

FIG. 1 shows the results of PCR analysis of transgenic pig (Tg pig). Lne M is the DNA ladder; Lane 1 is Tg pig 250-10; land 3 is Tg pig 250-12; Lane 5 is Tg ig 250-14; lane 10 is Tg pig 261-10; lane 14 is DNA control, lane N is non-transgenic control and lane W is water.

FIG. 2 shows Southern blot analysis for the estimation of the copy numbers of transgenic pigs. M1 is 1 kb step ladder marker; M2 is x/Hind III marker; 1 is 250-10; 2 is 250-12; 3 is 250-14; 4 is 260; 5 is 265-01; 6 is 265-13; 7 is 265-14; 8 is 292-01; 9 is 309-05; 10 is 3/9-10; 11 is 326-13; 12 is 329-10 (diluted 5 fold); 13 is 326-11; 14 is 326-12 (negative control); 15 is 5 copies of injected DNA fragments; 16 is 10 copies of injected DNA fragments.

### DETAILED DESCRIPTION

The invention relates to nucleic acids and viral vectors containing an enhancer with a mutated NF-E2/AP1 site (e.g., the HS-40(mt) enhancer), and their use in expressing RNA in an animal. Nucleic acids including the mtNF-E2/AP1 site can be used to form transgenic animals of the invention which express an antisense transcript or a mRNA encoding the protein to be expressed in the transgenic animal. The expression of the transgene is not affected by its position in the genome, nor is the expression inhibited at high transgene copy numbers (e.g., above 5, 7, 9, 14, or 19 copies). Instead, the expression level is directly correlated with transgene copy number, thereby allowing high levels of expression at high transgene copy numbers.

### PRODUCTION OF TRANSGENIC NON-HUMAN ANIMALS

In general, transgenic animal lines can be obtained by generating transgenic animals having incorporated into their genome at least one transgene, selecting at least one founder from these animals and breeding the founder or founders to establish at least one line of transgenic animals having the selected transgene incorporated into their genome.

Animals for obtaining eggs or other nucleated cells (e.g. embryonic stem cells) for generating transgenic animals can be obtained from standard commercial sources such as Charles River Laboratories (Wilmington, Mass.), Taconic (Germantown, N.Y.), Harlan Sprague Dawley (Indianapolis, Ind.).

Eggs can be obtained from suitable animals, e.g., by flushing from the oviduct or using techniques described in U.S. Pat. No. 5,489,742 issued Feb. 6, 1996 to Hammer and Taurog; U.S. Pat. No. 5,625,125 issued on Apr. 29, 1997 to Bennett et al.; Gordon et al., 1980, Proc. Natl. Acad. Sci. USA 77:7380-7384; Gordon & Ruddle, 1981, Science 214:1244-1246; U.S. Pat. No. 4,873,191 to T. E. Wagner and P. C. Hoppe; U.S. Pat. No. 5,604,131; Armstrong, et al. (1988) J. of Reproduction, 39:511 or PCT application No. PCT/FR93/00598 (WO 94/00568) by Mehtali et al., all of which are hereby incorporated by reference. Preferably, the female is subjected to hormonal conditions effective to promote superovulation prior to obtaining the eggs.

Many techniques can be used to introduce DNA into an egg or other nucleated cell, including in vitro fertilization using sperm as a carrier of exogenous DNA (“sperm-mediated gene transfer”, e.g., Lavitrano et al., 1989, Cell 57: 717-723), microinjection, gene targeting (Thompson et al., 1989, Cell 56: 313-321), electroporation (Lo, 1983, Mol. Cell. Biol. 3: 1803-1814), transfection, or retrovirus mediated gene transfer (Van der Putten et al., 1985, Proc. Nati. Acad. Sci. USA 82: 6148-6152). For a review of such techniques, see Gordon (1989), Transgenic Animals, Intl. Rev. Cytol. 115:171-229. All of their references are hereby incorporated by reference.

Except for sperm-mediated gene transfer, eggs should be fertilized in conjunction with (before, during or after) other transgene transfer techniques. A preferred method for fertilizing eggs is by breeding the female with a fertile male. However, eggs can also be fertilized by in vitro fertilization techniques.

Fertilized, transgene containing eggs can than be transferred to pseudopregnant animals, also termed “foster mother animals”, using suitable techniques. Pseudopregnant animals can be obtained, for example, by placing 40-80 day old female animals, which are more than 8 weeks of age, in cages with infertile males, e.g., vasectomized males. The next morning, females are checked for vaginal plugs. Females who have mated with vasectomized males are held aside until the time of transfer.

Recipient females can be synchronized, e.g. using GNRH agonist (GnRH-a): des-gly10, (D-Ala6)-LH-RH Ethylamide, SigmaChemical Co., St. Louis, Mo. Alternatively, a unilateral pregnancy can be achieved by a brief surgical procedure involving the “peeling” away of the bursa membrane on the left uterine horn. Injected embryos can then be transferred to the left uterine horn via the infundibulum. Potential transgenic founders can typically be identified immediately at birth from the endogenous litter mates. For generating transgenic animals from embryonic stem cells, see e.g. Teratocarcinomas and embryonic stem cells, a practical approach, ed. E. J. Robertson, (IRL Press 1987) or in Potter, et. al. Proc. Natl. Acad. Sci. USA 81, 7161 (1984), the teachings of which are incorporated herein by reference.

Founders that express the gene can then bred to establish a transgenic line. Accordingly, founder animals can be bred, inbred, crossbred or outbred to produce colonies of animals of the present invention. Animals comprising multiple transgenes can be generated by crossing different founder animals (e.g. an HIV transgenic animal and a transgenic animal, which expresses human CD4), as well as by introducing multiple transgenes into an egg or embryonic cell as described above. Furthermore, embryos from A-transgenic animals can be stored as frozen embryos, which are thawed and implanted into pseudo-pregnant animals when needed (See e.g. Hirabayashi et al. (1997) Exp Anim 46: 111 and Anzai (1994) Jikken Dobutsu 43: 247).

The present invention provides for transgenic animals that carry the transgene in all their cells, as well as animals that carry the transgene in some, but not all cells, i.e., mosaic animals. The transgene can be integrated as a single transgene or in tandem, e.g., head to head tandems, or head to tail or tail to tail or as multiple copies.

The successful expression of the transgene can be detected by any of several means well known to those skilled in the art. Non-limiting examples include Northern blot, in situ hybridization of mRNA analysis, Western blot analysis, immunohistochemistry, and FACS analysis of protein expression.

Transgenic Chickens

Transgenic chickens are made by procedures well known in the art. For example, Salter, et al., Virology 157:236-240 1987; Love, et al., bio/Technology 12:60-63 (1994); Crittendon, et al., J. Reprod. Fert. Suppl. 41:163-171 (1990); Carscience, et al., Development 117:669-675 (1993) the teachings of which are hereby incorporated by reference describe methods of producing transgenic chickens. In particular, transgenic chickens of the invention may be made by incorporating DNA constructs containing the HS-40(mt) enhancer into the genome of avian leukosis viruses and the viruses are injected near the blastoderm of fertile eggs prior to incubation. The embryo of a newly laid fertile egg is pluripotent and the injection of avian leukosis viruses near the embryo serves to infect some germ cells.

Transgenic Goats

Transgenic goats are produced by procedures well known in the art. Swiss origin goats, e.g. the Alpine, Saanen, and Toggenburg breeds are useful in the production of transgenic goats. Generally, the steps to produce transgenic goats include (1) superovulation of female goats; (2) mating to fertile males and (3) collection of fertilized embryos. Once collected, pronuclei of one-cell fertilized embryos are microinjected with DNA constructs containing the HS-40(mt) enhancer. Such procedures are detailed in U.S. Pat. Nos., 6,210,736; 6,183,803, 5,843,705 and 5,827,690 all of which are hereby incorporated by reference.

Transqenic Sheep

Transgenic sheep are made by procedures well known in the art. A protocol for the production of a transgenic sheep can be found in Transgenic Animal Technology, A Handbook, 1994, ed. Carl A. Pinkert, Academic Press, Inc., which is hereby incorporated by reference. DNA constructs containing the HS-40(mt) enhancer are introduced into sheep by these procedures.

Transgenic Cows

Transgenic cows are made by procedures well known in the art. A protocol for the production of transgenic cows can be found in Transgenic Animal Technology, A Handbook, 1994, ed. Carl A. Pinkert, Academic Press, Inc., which is hereby incorporated by reference. DNA constructs containing the HS-40(mt) enhancer are introduced into cows using these procedures.

Transgenic Rats

Transgenic rats are made by procedures well known in the art. A protocol for the production of a transgenic rat can be found in Bader and Ganten, Clinical and Experimental/Pharmacology and Physiology, supp. 3:581-587, 1996 which is hereby incorporated by reference. DNA constructs containing the HS-40(mt) enhancer are introduced into rats using these procedures.

Transgenic Rabbits

Transgenic rabbits are made by procedures well known it the art such as those described in PCT application No. PCT/FR93/00598 which is hereby incorporated by reference. DNA constructs containing the HS-40(mt) enhancer are introduced into rabbits using these procedures.

Production of Transgenic Mice

Transgenic mice are produced by procedures well known in the art. In the present invention, transgenic mice were produced by microinjection of DNA fragments into the pronuclei of fertilized mouse eggs as described in Brinster et al., Cell 27:223-231, 1981 and Costantini et al., Nature 294:92-94,1981. Plasmids pHS40-ζ597-GH and pHS40(r-mt 1)-ζ597-GH are described in Zhang et al., J. Biol. Chem. 270:8501-8505 (1995). Digestion of these plasmids with EcoRi, Ndel, and Scal yielded 3.12 kb DNA fragments containing the HS40 enhancer, the ζ-globin promoter, and the growth hormone (GH) open reading frame. The 3.12 kb DNA fragments were eluted from soft agarose gels, purified, and used for microinjection.

Transgenic founders were identified and their transgene copy number determined by Southern blot analysis of tail DNA. The founders were then bred with nontransgenic C57/B6 mice to establish lines. The morning on which the copulatory pluf was observed was designated 0.5 day postcoital. For analysis of fetal (14.5 days postcoital) and embryonic (9.5 days postcoital) mice, transgenic males were mated to nontransgenic C57/B6 females. Transgenic pups were identified by PCR analysis of fetal mice tails or of embryo DNA. For each identification, duplicate PCR reactions were carried out using one 5′ primer from the ζ-globin promoter region, and two different 3′ primers from the GH region (see below).

A total of 9 founders with wild type HS-40(wt)-597GH and 10 founders with the mutant HS-40(mt)-5GH have been obtained. The copy numbers of integrated fragments in HS-40(wt)-containing mice vary from 1 to more than 100, as shown in Table 1.

TABLE 1

| Mutant HS-40 Transgene | | | Wild Type HS-40 Transgene | | |
|---|---|---|---|---|---|
| Founder | Copy | hGH, | Founder | Copy | hGH, |
| line | number | ng/ml | line | number | ng/ml |
| 1A* | 1 | 470 | 1A* | 1 | 36 |
| 1B* | 1 | 530 | 1B* | 1 | 20 |
| 1C* | 1 | 1,060 | 2 | 2 | 14 |
| 2 | 2 | 650 | 3 | 3 | 22 |
| 3 | 3 | 1,260 | 5 | 5 | 5 |
| 8* | 8 | 2,990 | 10* | 10 | 13 |
| 10* | 10 | 3,360 | 13* | 13 | 187 |
| 13* | 13 | 4,650 | 100A | >100 | 1,400 |
| 15* | 15 | 5,560 | 100B | >100 | 30 |
| 19* | 19 | 6,490 | | | |

In Table 1, the founders for which lines have been established are indicated by an asterisk. Mice with the HS-40(wt) transgene were assayed at the age of 5 months except founder 1B, which was evaluated at 9 months old. Mice with the HS-40(mt) transgene were assayed at the age of 4 months except founder 15, which was evaluated at 2 months old.

The ζ-globin promoter activities in the founder mice were first measured with a blood GH assay as described in Zhang et al., supra. The levels of human GH in the blood were quantitated with the Allegro hGH radioimmunoassay kit from Nichols Institute. When the concentration of GH in the blood exceeded 50 ng/ml, the samples were first diluted with horse serum in order perform the assay in a linear range.

It was known that the amount of secreted enzyme molecules are good representations of the quantities of mRNAs inside the expressing cells (Zhang et al., supra; Palmiter et al., Nature 300:611-615, 1982; Palmiter et al., Science 222:809-814, 1983; Hammer et al., Nature 315:680-683, 1985; and Selden et al., Mol Cell Biol 6:3137-3179, 1986). The level of GH in HS-40(wt) transgenic mice were all low and comparable to non-transgenic controls. This was consistent with observations that the human ζ-globin promoter activity is essentially shut off in adult transgenic mice, even when it is linked in cis with the HS-40(wt) enhancer or with the β-globin locus control region (Pondel et al., Nucl Acids Res 20:5655-5660, 1992; Robertson et al., Proc Natl Acad Sci USA 92:5371-5375, 1995; Albitar et al., Mol Cell Biol 11:3786-3794, 1991; and Spanger et al., Nucl Acids Res 18:7093-7097, 1990).

In contrast, the blood GH levels of the ten founder mice having the HS-40(mt) enhancer exhibited a roughly linear, positive relationship relative to transgene copy number. Further, the expression of the HS-40(mt) transgene was integration site-independent (i.e., position-independent) because the integration sites here were believed to be random and mice having similar transgene copy numbers exhibit similar level of expression. The blood GH levels these founders at other ages, as well as these founders' progeny, were similar to the levels of expression in HS-40(mt)-containing mice, as shown in Table 1.

To analyze the GH RNA levels in transgenic fetuses and embryos, liquid N₂-frozen embryos, fetuses, or fetal livers were manually homogenized, and the RNA isolated by standard acid guandinium isothiocyanate-phenol-chloroform extraction (Sambrook et al., Molecular Cloning: A Laboratory Manual, Cold Spring Harbor Laboratory Press, Plainview, N.Y., 2nd ed., 1989). For adult samples, the mice were rendered anemic by three injections of phenylhydrazine (40 μg/g of body weight) so that erythroblasts would enter the adult blood and be collected for analysis. The second injection was 8 hours after the first injection, and the third injection was 24 hours after the first. Six days after the first injection, the mice were sacrificed, and the RNA was isolated from different tissues. In all cases, the total RNA was used for the following assay without further purification.

RT-PCR was carried out as described in Chelly et al., Nature 333:858-860,1988 and Foley et al., Trends Genet 9:380-385, 1993. Each reverse transcription reaction mixture contained 1 μg of RNA, 200 units of SUPERSCRIPT Il® reverse transcriptase (Gibco BRL), and 20 mM oligo d(T)₁₅. One-twentieth of the cDNA was then amplified by PCR using Taq polymerase (Gibco BRL) and primers specific for human GH, mouse β^{major}, mouse 70 -globin promoter, or mouse G3PDH. Amplifications were carried out in a HYBRID OmniGene system with the following temperature profiles: an initial denaturation at 95° C. for 3 min, 53° C. for 1 min, and 72° C. for 1 min; followed by repeating cycles of 95° C. for 1 min, 53° C. for 1 min, and 72° C. for 1 min; and finally an elongation step at 72° C. for 5 min. Each PCR analysis was done in duplicate. The sequences of PCR primers used are as follows. For mG3PDH, TGMGGTCGGTGTGAACGGATTTGGC (SEQ ID NO:4) was used as the 5′ primer, and CATGTAGGCCATGAGGTCCACCAC (SEQ ID NO:5) was used at the 3′ primer. For the human GH gene, GTCCCTGCTCCTGGCTTT (SEQ ID NO:6) was used as the 5′ primer, and ATGCGGAGCAGCTCCAGGTT (SEQ ID NO:7) was used as the 3′ primer. Another 3′ primer used for the human GH gene was CATCAGCGTTTGGATGCCTT (SEQ ID NO:8). For the mouse β^{major} sequence, TGGGCAGGCTGCTGGTTA (SEQ ID NO:9) was used at the 5′ primer, and TTAGTGGTACTTGTGAGCCAA (SEQ ID NO:10) was used as the 3′ primer. For the mouse ζ-globin promoter sequence, CTGATGMGAATGAGAGAGC (SEQ ID NO:11) was used as the 5′ primer, and TAGAGGTACTTCTCATCAGTCAG (SEQ ID NO:12) was used as the 3′ primer. The PCR product lengths were 980 bp for mouse G3PDH, 335 bp for mouse β^{major}, and 290 bp or 450 bp for ζ-GH. One-fifth of each PCR reaction was resolved on a 1.5% agarose-ethidium bromide gel, which was then documented using a IS1000 Digital Imaging System and saved as a TIF computer file. The band intensities were quantitated by the Phosphorlmage System.

For semi-quantitative purposes, mouse G3PDH was used as the internal standard. The linearity of amplification of the G3PDH cDNA was first defined by amplification of serial dilutions of the cDNA samples. Twenty five cycles were chosen for amplifying mouse G3PDH since, under the reaction conditions described above, the signals were linear over a wide range of dilutions of cDNA. In the initial calibration test, G3PDH bands with similar intensities were obtained from the different tissue CDNA when the same amount of RNA was used for reverse transcription. The appropriate PCR cycle number used to amplify the human GH, mouse β^{major}, and mouse ζ-globin transcripts were 28, 25 and 28, respectively. The amount of different cDNA used for amplification were first determined by PCR using the mouse G3PDG primers, then individual PCR reactions were performed using the human GH, mouse β^{major}, or mouse ζ-globin primers.

It was known that, in the developing mouse, the first site of erythropoiesis is at the yolk sac blood island at 8-14 days of gestation. The major site of erythropoiesis then shifts to the fetal liver, and finally to the spleen at birth. The expression of GH transcripts from the mouse ζ-globin promoter in adult transgenic mice containing the HS40(mt) enhancer was examined. In all adult mice having the HS-40(mt) transgene, the expression of GH RNA was restricted to the erythroid tissues. Expression was roughly limited to the spleen and blood, with no expression in the liver or brain. Expression could not be detected in the blood of mice containing the HS-40(mt) transgene unless the mice were first rendered anemic, indicating that expression was erythroblasts-specific. Mice having the HS-40(wt) transgene exhibited little, if any, expression.

The expression of the transgenic mice at the fetal stage also appeared to be erythroid-specific. ζ-GH transcripts could be detected in 14.5 day fetuses from transgenic mice with either HS-40(mt) or HS-40(wt) sequences. No ζ-GH transcripts were detected in non-transgenic control mice. A high intensity RT-PCR band was apparent in the reaction containing fetal liver RNA, consistent with the erythroid fetal liver being the major site of transcription of ζ-GH transgenes.

Changes in ζ-GH transgene expression were followed by RT-PCR. Transgenic mice having the HS-40(wt) transgene exhibited the expected temporal pattern of expression during development, the level of ζ-GH transcripts was relatively high at the 9.5 day embryo stage but dropped significantly in the adult blood. In contrast, the transgenic mice having the HS-40(mt) enhancer continued to express the ζ-GH transcript into adulthood. In addition, even with only one copy of the transgene, mice having the HS-40(mt) expressed at a higher level than mice having the HS-40(wt) enhancer, regardless of the stage of development.

These data indicated that the HS-40(mt) enhancer sequence not only relieved the repression of the ζ-globin promoter in adulthood, but enhanced expression at all stages of development, even at one transgene copy per genome. When combined with the linear relationship between transgene copy number and expression level, as described above, the results indicated that HS-40(mt) can be used as an enhancer of gene expression in a variety of contexts.

Production of Transgenic Pigs

A. Materials And Methods

Transgenic pigs are produced by procedures well known in the art. In the present invention, pure breed Landrace (L), Yorkshire (Y), or Duroc (D) gilts were used in this experiment and were fed 1.0 to 1.2 kg commercial ration twice per day and water were ad libitum provided. Sows in lactation were given lactation diet and access to water freely. The transgenic piglets were weaned at 28 days after delivery. All embryos' donor and recipient gilts were synchronized by feeding Regumate® (containing 0.4% altrenogest, 20 mg/day; Hoechst, France), which were mixed with commercial feed in the morning for 15 days. They were superovulated by injection of PMSG (1,500 or 2,000 IU, i.m.) 24 hrs after the last feeding Regumate®, and by injection of hCG (1,250 or 1,750 IU, i.m.) 76 to 78 hrs after the injection of PMSG. Finally, they were mated by artificial insemination twice with pure breed boars' fresh-diluted semen at 24 and 36 hr after the injection of hCG. Fifty-six to sixty hours after hCG injection, the donor pigs were surgically operated to flush fertilized eggs from the Fallopian tubes with 20 ml Dulbecco's PBS with 0.1% BSA into a dish. Before operation, the pigs were fasted overnight, tranquilized by injection (i.m.) of 5 ml sterinil and 10 ml atropine sulfate, and anaesthetized by injection of sodium pentobarbitone into an ear vein. Anaesthesia was maintained throughout the operation via a closed-circuit system using 4% halothene (ICI, Ltd) in an oxygen gas mixture. After the embryos' manipulation was completed, the DNA-injected pig embryos were transferred into the Fallopian tube of other synchronized foster pigs by surgical methods with the same procedures as for donors. Until piglets were delivered, the piglets' ear or tail's tissues were taken to extract their genomic DNA for analysis.

For gene transfer, HS40(mt)-β597-hGH DNA for injection was diluted to 2 ng/ul in T.E. (10 mM Tris.HCl, 0.1 mM EDTA, pH=7.8) buffer. The fertilized eggs were centrifuged with 22,000 g for 10 min at room temperature (Hettich EBA 12, Germany) to expose the pronuclei. Pigs' embryos were micromanipulated by Leica mechanical manipulator (Leica, Germany) with differential interference contrast inverted microscope (ZEISS Axiovert 135, Germany). The DNA fragments were injected into male pronuclei of the pig embryos. Sets of 20 embryos were transferred into the Fallopian tubes of synchronized-recipients immediately following injections.

After pregnancy of sow completed, the piglets were delivered and their birth weights were determined. DNA were extracted from the ears of live piglets or the tail tissues of the stillborn piglets on the delivery day. The transgenes were assayed by PCR with the primer pair of: 5′- CTG ATA AGA MC ACC ACC CC-3′ (SEQ ID NO: 13) and 5′-ATG CGG AGC AGC TCC TGG TT-3′ (SEQ ID NO: 14). The PCR conditions were 94° for 3 min and 40 cycles of amplification, each cycle with denaturion at 94° for 1 min, annealing at 55° for 1 min, and extension at 73° for 1 min. The PCR products were analyzed by 2% agarose gel electrophoresis for a 1070 bp band of the positives.

For Southern blot analysis, the genomic DNAs (10 pg) were digest with Eco RI, separated by 0.8% agarose gel electrophoresis, blotted onto nylon membrane, and hybridized with P³²-labeled HS40(mt)-ζ597 probe. The copy numbers of the transgenic was estimated by comparison of the signals to know quantities of DNA on the same membrane.

Growth hormone was assayed in the following manner. On and after 7 days of the delivery, 4 ml of blood samples of each transgenic piglet were taken via direct needle-punch at the superior vena cava. 2 mls of blood were assayed for RT-PCR analysis, and 2 ml were assayed for the hGH levels by ELISA.

Pigs were analyzed with a growth performance test. All piglets were weighed on the day of birth, at weaning, and after 11 weeks. The growth performance test was conducted in the farm. That data regarding the dairy gain, feed efficiency, back fat, and area of lion were collected assessed. The day periods from birth to reach alive weight of 108-110 kg were also recorded.

B. Results And Discussion

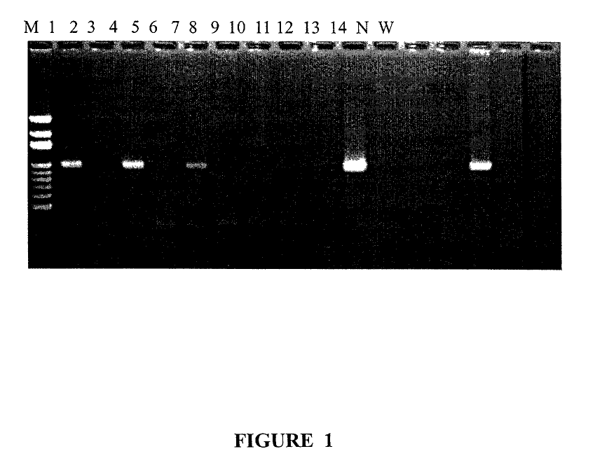

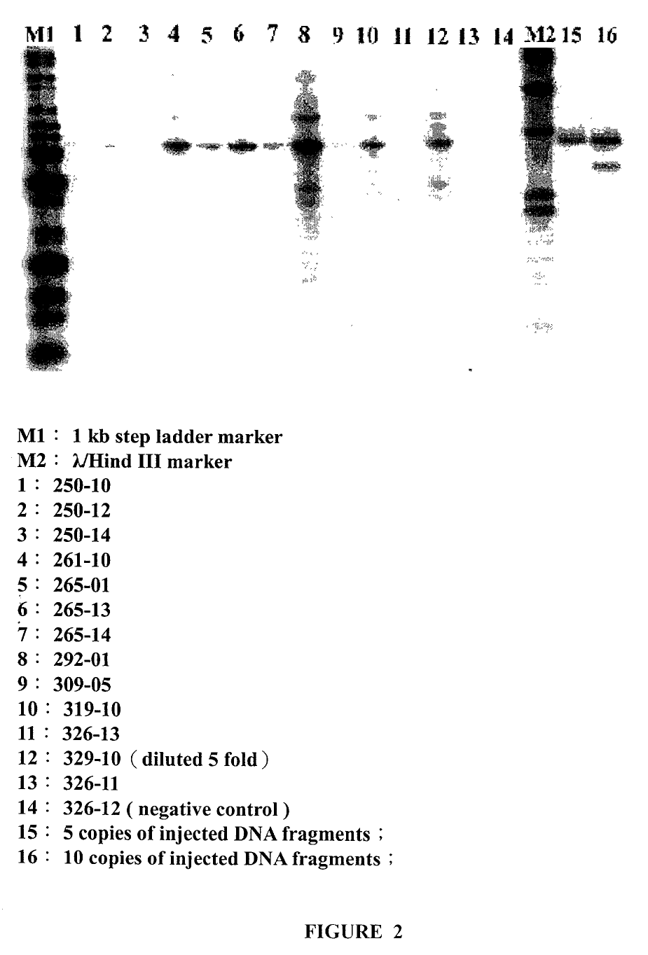

A total of 692 pig embryos were microinjected with the transgene fragment, and then transferred to 26 surrogate sows, of which 17 (65.4%) were pregnant and farrowed 113 piglets including the stillborn ones. PCR analysis showed that there were 19 transgenic piglets proved (FIG. 1), and 6 of them were still alive (Table 2.) The birth weights of the transgenic piglets were from 0.6 to 1.8 kg with an average of 1.08 kg, which was not heavier than the average weight of the non-transgenic piglets (1.37 kg) or the total littermate (1.34 kg), as observed in the transgenic mice studies described above. The birth weights also did not correlate with the copy numbers (FIG. 2 and Table 3). However, the blood hGH levels of the transgenic pigs 326-13, 250-12, 265-01, and 329-10 are 155, 675, 1,525, and 53,800 ng/ml, respectively, which correlate with their transgene copy numbers (Table 4).

The assessment of the growth performance of these transgenic pigs is continually monitored. GH is physiologically secreted in a pulsatile pattern with an interval about 6 h in normal pigs. However, in transgenic pigs, the hGH was sustained with a high plateau pattern. This abnormality of hGH secretion pattern may be attributed to the weakness and/or death (Table 3) of some of the transgenic pigs. Reproductive defects of transgenic pig expressing bovine growth hormones have been observed before by others (Guthrie et al., 1993). In this study, all alive transgenic pigs are still under close observation.

C. Assessment Of Results

(1) There were 19 transgenic piglets obtained, among which 7 were stillborn at the delivery or died during nursing period because of weakness, 3 transgenic piglets were weak and crushed to death by sows, 3 transgenic pigs were lost due to disease infection, and 6 transgenic piglets remained alive and have all given birth to a second generation of piglets. We suspected that transgenic pigs continually expressing high level of hGH may have interrupted physiological homeostasis leading to the relatively high numbers of stillborn and weak transgenic pigs.

(2) The levels of expression of hGH in the transgenic pigs correlated with the increase of the copy numbers of the transgene. Also, they appeared to be independent of the transgenic lines. The data showed that the novel expression cassette, HS40(mt)-ζ597, indeed could perform well as a good molecular vector to drive transgenic expression for the production of medical proteins in domestic animals.

(3) The birth weights of the transgenic piglets were not heavier than the non-transgenic pigs. Tests on the farm can show whether the growth ance including the daily gain, feed efficiency, surface area of lion, back fat, icted age of 110 kg can be improved by the expression of hGH.

(4) Other medically important proteins can be used as candidates for on in transgenic mice models and in transgenic domestic animals. Such dically important proteins include those described in PCT publication WO 00/69900 which has been incorporated by reference.

TABLE 2
Generation of HS40(mt)-ζ597-hGH transgenic pigs
by microinjection of pronuclei of pig zygotes

| No. of | No. of embryo | No. of recipient | | No. of piglet | |
|---|---|---|---|---|---|
| experiment | microinjected | E.T. Delivered % | | Born | Transgenic % |
| 10 | 423 | 17 | 12 (70.6) | 90 | 2/10^{a} (13.7) |
| 5 | 258 | 9 | 5 (50.0) | 41 | 4/3^{a} (17.1) |
| 15 | 692 | 26 | 17 (65.4) | 131 | 7/12^{a} (14.5) |

^{a}Live/die transgenic piglets.

TABLE 3
Birth weights (BWt) and transgene copy numbers
of the transgenic (Tg) pigs

| Tg pig | BWt | Litter mate | | Copy no. of | |
|---|---|---|---|---|---|
| lines | kg | Total | Avg. BWt kg | transgene | Remarks |
| 250-10 | 1.2 D | 9 | 1.68 (1.2-2.0)* | 2 | crushed |
| 250-12 | 1.5 L | 9 | 1.68 (1.2-2.0) | 2 | |
| 250-14 | 1.8 D | 9 | 1.68 (1.2-2.0) | 1 | crushed |
| 261-10 | 0.7 D | 7 | 1.06 (0.7-1.4) | 5 | Weak and |
| | | | | | crushed |
| 265-01 | 0.7 D | 11 | 0.81 (0.4-1.2) | 2 | Infection |
| 265-13 | — D | 11 | 0.81 (0.4-1.2) | 4 | Stillborn |
| 265-14 | — D | 11 | 0.81 (0.4-1.2) | 2 | Stillborn |
| 292-01 | 1.5 D | 6 | 1.77 (1.5-2.0) | 9 | Infection |
| 309-05 | 0.6 D | 12 | 1.20 (0.6-1.5) | 1 | Weak and die |
| 319-10 | 0.9 D | 9 | 1.54 (0.9-1.9) | 3 | Weak and |
| | | | | | crushed |
| 326-13 | 1.8 L | 5 | 1.74 (1.1-2.2) | 1 | |
| 329-10 | 1.3 L | 6 | 1.52 (1.1-1.9) | 20 | Infection |
| 036-14 | 1.0 D | 8 | 1.16 (0.7-1.6) | — | crushed |
| 036-15 | 1.2 L | 8 | 1.16 (0.7-1.6) | — | |
| 036-F | 1.2 D | 8 | 1.16 (0.7-1.6) | — | Stillborn |
| 036-M | 0.7 D | 8 | 1.16 (0.7-1.6) | — | Stillborn |
| 049-01 | 1.0 L | 10 | 1.30 (0.9-1.6) | — | |
| 82-03 | 1.3 L | 10 | 1.46 (1.1-1.8) | — | |
| 82-10 | 1.1 L | 10 | 1.46 (1.1-1.8) | — | |
| Avg. | 1.08 | 65 | 1.37 | | |

D-dead
L-live
— not done

TABLE 4
Growth hormone assay of transgenic pigs

| Line | Copy No. | Growth hormone (ng/ml) |
|---|---|---|
| 036-11 | Negative | 0 |
| 326-13 | 1 | 155 |
| 250-12 | 2 | 675 |
| 265-01 | 2 | 1,525 |
| 329-10 | 20 | 53,800 |

1. A transgenic non-mouse non-human animal whose somatic and germ line cells contain at least one copy of a transgene comprising (1) a transcriptional start site; (2) a promoter operably linked to the transcriptional start site; and (3) an enhancer operably linked to the promoter, the enhancer comprising the nucleotide sequence of SEQ ID NO:1,
wherein the transgenic animal expresses a transcript driven by the promoter, the level of expression in at least one cell type of the animal being proportionally dependent on the copy number of the transgene.

2. The transgenic animal of claim 1, wherein the animal is selected from the group consisting of pig, rat, cow, rabbit, goat, guinea pig, prarie baboon, squirrel, monkey, chimpanzee, bird, frog, toad, chicken, turkey and sheep.

3. The transgenic animal of claim 2, wherein the animal is a pig.

4. The transgenic animal of claim 3, wherein the somatic and germ line cells contain more than 5 copies of the transgene.

5. The transgenic animal of claim 4, wherein the somatic and germ line cells contain more than 15 copies of the transgene.

6. The transgenic animal of claim 5, wherein the promoter drives transcription of a mRNA encoding a polypeptide, the transcription beginning from the transcriptional start site.

7. The transgenic animal of claim 6, wherein the polypeptide is a growth hormone.

8. The transgenic animal of claim 7, wherein the promoter is a ζ-globin promoter, and the at least one cell type is a erythroblast.

9. The transgenic animal of claim 8, wherein the enhancer comprises SEQ ID NO:2.

10. The transgenic animal of claim 9, wherein the enhancer comprises SEQ ID NO:3.

11. A method of expressing a transcript in a non-mouse, non-human animal, the method comprising administering to the animal a nucleic acid comprising (1) a transcriptional start site for the transcript; (2) a promoter operably linked to the transcriptional start site; and (3) an enhancer operably linked to the promoter, the enhancer comprising the DNA sequence of SEQ ID NO:1 or the RNA equivalent thereof.

12. The method of claim 11, wherein the animal is selected from the group consisting of pig, rat, cow, rabbit, goat, guinea pig, prarie baboon, squirrel, monkey, chimpanzee, bird, frog, toad, chicken, turkey and sheep.

13. The method of claim 11, wherein the nucleic acid is administered via a viral expression vector.

14. The method of claim 11, wherein the transcript is a mRNA encoding a polypeptide.

15. The method of claim 14, wherein the polypeptide is a growth hormone.

16. The method of claim 11, wherein the promoter is a ζ-globin promoter.

17. The method of claim 11, wherein the enhancer comprises SEQ ID NO:2 or the RNA equivalent thereof.

18. The method of claim 11, wherein the enhancer comprises SEQ ID NO:3 or the RNA equivalent thereof.

19. The method of claim 11, wherein the nucleic acid further comprises a transcriptional termination signal.

20. The method of claim 19, wherein the transcriptional termination signal is a polyadenylation signal.
